(19) United States
(12) Patent Application Publication
(10) Pub. No.: US 2011/0160785 A1
(43) Pub. Date: Jun. 30, 2011

(54) DEFIBRILLATION CATHETER

(76) Inventors: Kenji Mori, Tokyo (JP); Yasuo Sakano, Tokyo (JP)

(21) Appl. No.: 12/998,008

(22) PCT Filed: Aug. 24, 2009
(86) PCT No.: PCT/JP2009/064693

§ 371 Date: Mar. 7, 2011

(30) Foreign Application Priority Data
Sep. 11, 2008 (JP) 2008-233742

(51) Int. Cl. A61N 1/39

(52) U.S. Cl. 607/5

(57) ABSTRACT

An intracardiac defibrillation catheter capable of supplying electric energy necessary and sufficient for defibrillation and conducting a defibrillation treatment without causing a burn on the body surface of a patient. The intracardiac defibrillation catheter is equipped with an insulated tube member having a multi-lumen structure, a handle connected to a proximal end of the tube member, a DC electrode group installed in a distal region of the tube member, a second DC electrode group installed on the tube member towards proximal direction from the first DC electrode group, a first lead wire group composed of lead wires connected to the respective electrodes making up the first DC electrode group, and a second lead wire group composed of lead wires connected to the respective electrodes making up the second DC electrode group, wherein the first lead wire group and the second lead wire group respectively extend into different lumens of the tube member.

### TECHNICAL FIELD

The present invention relates to an intracardiac defibrillation catheter inserted into a cardiac cavity to remove atrial fibrillation.

### BACKGROUND ART

An automated external defibrillator (AED) is known as a defibrillator for removing atrial fibrillation (see, for example, Patent Literature 1).

In a defibrillation treatment by the AED, an electrode pad is attached to the body surface of a patient to apply a direct current voltage thereto, thereby giving electric energy within the body of the patient. Here, the electric energy flowing within the body of the patient from the electrode pad is generally controlled to 150 to 200 J, and a part (generally, about several % to 20%) thereof flows into a heart to be used for defibrillation treatment.

### PRIOR ART LITERATURE

### Patent Literature

Patent Literature 1: Japanese Patent Application Laid-Open No. 2001-112874

### SUMMARY OF THE INVENTION

### Problem to be Solved by the Invention

And now, the atrial fibrillation is liable to occur during cardiac catheterization, and it is necessary to conduct electrical defibrillation even in this case.

According to the AED that electric energy is supplied from the outside of the body, however, it is difficult to supply effective electric energy (for example, 10 to 30 J) to a heart that has suffered the atrial fibrillation.

In other words, when a proportion of electric energy flowing into the heart of the electric energy supplied from the outside of the body is small (for example, about several %), it is impossible to conduct a sufficient defibrillation treatment.

On the other hand, when the electric energy supplied from the outside of the body flows into the heart in a high proportion, it is considered that the tissue of the heart may possibly be damaged.

In addition, in the defibrillation treatment by the AED, burn is easy to occur on the body surface to which the electrode pad has been attached. When the proportion of the electric energy flowing into the heart is small as described above, the degree of burn becomes heavy by supplying the electric energy repeatedly to be a considerable burden on the patient subjected to the catheterization.

The present invention has been made on the basis of the foregoing circumstances and has as an object the provision of an intracardiac defibrillation catheter capable of surely supplying electric energy necessary and sufficient for defibrillation to a heart that has suffered atrial fibrillation during cardiac catheterization.

Another object of the present invention is to provide an intracardiac defibrillation catheter by which a defibrillation treatment can be conducted without causing burn on the body surface of a patient.

### Means for Solving Problem

(1) The intracardiac defibrillation catheter according to the present invention is a catheter inserted into a cardiac cavity to conduct defibrillation, the catheter comprising

an insulated tube member having a multi-lumen structure,

a handle connected to a proximal end of the tube member,

a first electrode group (a first DC electrode group) composed of a plurality of ring-like electrodes and installed in a distal region of the tube member,

a second electrode group (a second DC electrode group) composed of a plurality of ring-like electrodes and installed on the tube member towards proximal direction (with a space on a proximal side) from the first DC electrode group,

a first lead wire group composed of lead wires connected to the respective electrodes making up the first DC electrode group, and

a second lead wire group composed of lead wires connected to the respective electrodes making up the second DC electrode group, wherein

the first lead wire group and the second lead wire group respectively extend into different lumens of the tube member, and

voltages different in polarity from each other are respectively applied to the first DC electrode group and the second DC electrode group when defibrillation is conducted.

The intracardiac defibrillation catheter of such a structure is inserted into a cardiac cavity in such a manner that the first DC electrode group is located in a coronary vein, and the second DC electrode group is located in a right atrium to respectively apply voltages different in polarity from each other to the first DC electrode group and the second DC electrode group (apply a direct current voltage between the first DC electrode group and the second DC electrode group) through the first lead wire group and the second lead wire group, thereby directly applying electric energy to a heart that has suffered fibrillation to conduct a defibrillation treatment.

As described above, the electric energy is directly given to the heart that has suffered the fibrillation by the first DC electrode group and the second DC electrode group of the defibrillation catheter arranged within the cardiac cavity, whereby electric stimulus (electric shock) necessary and sufficient for defibrillation treatment can be surely given only to the heart.

In addition, no burn is caused on the body surface of the patient because the electric energy can be directly applied to the heart.

The first lead wire group composed of lead wires connected to the respective electrodes making up the first DC electrode group, and the second lead wire group composed of lead wires connected to the respective electrodes making up the second DC electrode group respectively extend into the different lumens of the tube member, whereby both lead wire groups are completely insulated and isolated from each other within the tube member. Thus, occurrence of short circuit between the first lead wire group (first DC electrode group) and the second lead wire group (second DC electrode group) within the tube member can be surely prevented when the voltage necessary for the intracardiac defibrillation is applied.

(2) In the intracardiac defibrillation catheter according to the present invention, it may be preferable that the catheter comprises

a potential-measuring electrode group composed of a plurality of electrodes installed on the tube member apart from the first DC electrode group or the second DC electrode group, and

a lead wire group for potential measurement, which is composed of lead wires connected to the respective electrodes making up the potential-measuring electrode group, wherein

the lead wire group for potential measurement extends into any other lumen of the tube member than both lumens into which the first lead wire group and the second lead wire group extend.

According to the intracardiac defibrillation catheter of such a structure, a cardiac potential can be measured by the potential-measuring electrode group to conduct the defibrillation treatment while monitoring the cardiac potential.

The lead wire group for potential measurement extends into any other lumen of the tube member than both lumens into which the first lead wire group and the second lead wire group extend, whereby the lead wire group for potential measurement is completely insulated and isolated from both the first lead wire group and the second lead wire group. Thus, occurrence of short circuit between the lead wire group for potential measurement (potential-measuring electrode group) and the first lead wire group or second lead wire group (first DC electrode group or second DC electrode group) within the tube member can be surely prevented when the voltage necessary for the intracardiac defibrillation is applied.

(3) In the intracardiac defibrillation catheter according to the present invention, it may be preferable that the catheter comprises

a distal-side potential-measuring electrode group composed of a plurality of electrodes and installed on the tube member towards distal direction from the first DC electrode group,

a proximal-side potential-measuring electrode group composed of a plurality of ring-like electrodes and installed on the tube member towards proximal direction from the second DC electrode group,

a third lead wire group composed of lead wires connected to the respective electrodes making up the distal-side potential-measuring electrode group, and

a fourth lead wire group composed of lead wires connected to the respective electrodes making up the proximal-side potential-measuring electrode group, wherein

the third lead wire group and the fourth lead wire group extend into any other lumen of the tube member than both lumens into which the first lead wire group and the second lead wire group extend.

According to the intracardiac defibrillation catheter of such a structure, a cardiac potential can be measured by the distal-side potential-measuring electrode group and the proximal-side potential-measuring electrode group to conduct the defibrillation treatment while monitoring the cardiac potential.

The third lead wire group and the fourth lead wire group extend into any other lumen of the tube member than both lumens into which the first lead wire group and the second lead wire group extend, whereby the third lead wire group and the fourth lead wire group are completely insulated and isolated from both the first lead wire group and the second lead wire group. Thus, occurrence of short circuit between the third lead wire group or fourth lead wire group (distal-side potential-measuring electrode group or proximal-side potential-measuring electrode group) and the first lead wire group or second lead wire group (first DC electrode group or second DC electrode group) within the tube member can be surely prevented when the voltage necessary for the intracardiac defibrillation is applied.

(4) In the intracardiac defibrillation catheter of (3), it may be preferable that

4 lumens are formed in the tube member,

the first lead wire group extends into a first lumen,

the second lead wire group extends into a second lumen,

the third lead wire group and fourth lead wire group extend into a third lumen, and

a pull wire for distal end deflection operation extends into a fourth lumen.

According to the intracardiac defibrillation catheter of such a structure, occurrence of short circuit among the first lead wire group (first DC electrode group), the second lead wire group (second DC electrode group) and the third lead wire group or fourth lead wire group (distal-side potential-measuring electrode group or proximal-side potential-measuring electrode group) can be surely prevented. Since the pull wire for distal end deflection operation extends into any other lumen than the lumens into which the respective lead wire groups extend, the lead wires making up the lead wire groups are not damaged (for example, abraded) by the pull wire moving in an axial direction upon a distal end deflection operation.

(5) In the intracardiac defibrillation catheter according to the present invention, it may be preferable that the first lead wire group and the second lead wire group extend into inner holes of insulated tubes different from each other in the interior of the handle.

According to the intracardiac defibrillation catheter of such a structure, the first lead wire group and the second lead wire group are completely insulated and isolated from each other even in the interior of the handle. Thus, occurrence of short circuit between the first lead wire group and the second lead wire group in the interior of the handle can be surely prevented when the voltage necessary for the intracardiac defibrillation is applied.

(6) In the intracardiac defibrillation catheter of (4), it may be preferable that in the interior of the handle,

the first lead wire group extends into a first insulated tube connected to the first lumen,

the second lead wire group extends into a second insulated tube connected to the second lumen, and

the third lead wire group and the fourth lead wire group extend into a third insulated tube connected to the third lumen.

According to the intracardiac defibrillation catheter of such a structure, the first lead wire group, the second lead wire group, and the third lead wire group and the fourth lead wire group are completely insulated and isolated from one another even in the interior of the handle. Thus, occurrence of short circuit among the first lead wire group, the second lead wire group and the third lead wire group or the fourth lead wire group in the interior of the handle can be surely prevented when the voltage necessary for the intracardiac defibrillation is applied.

(7) The intracardiac defibrillation catheter according to the present invention may preferably be inserted into a cardiac cavity to remove atrial fibrillation caused during cardiac catheterization.

### EFFECTS OF INVENTION

According to the intracardiac defibrillation catheter of the present invention, electric energy necessary and sufficient for defibrillation can be surely supplied to a heart that has suffered atrial fibrillation during cardiac catheterization. In addition, no burn is caused on the body surface of a patient, and invasiveness is also little.

Further, occurrence of short circuit between the first lead wire group (first DC electrode group) and the second lead wire group (second DC electrode group) can be surely prevented when the voltage necessary for the intracardiac defibrillation is applied.

### BRIEF DESCRIPTION OF THE DRAWINGS

FIG. 1 is an explanatory plan view illustrating an embodiment of an intracardiac defibrillation catheter according to the present invention.

FIG. 2 is an explanatory plan view (drawing for explaining dimensions and hardnesses) illustrating the embodiment of the intracardiac defibrillation catheter according to the present invention.

FIG. 3 is a cross-sectional view illustrating a section A-A in FIG. 1.

FIG. 4 is cross-sectional views illustrating sections B-B, C-C and D-D in FIG. 1.

FIG. 5 is a longitudinal sectional view (illustrating a section E-E in FIG. 3) in a distal region of a multi-lumen tube.

FIG. 6 is a longitudinal sectional view (illustrating a section F-F in FIG. 3) in the distal region of the multi-lumen tube.

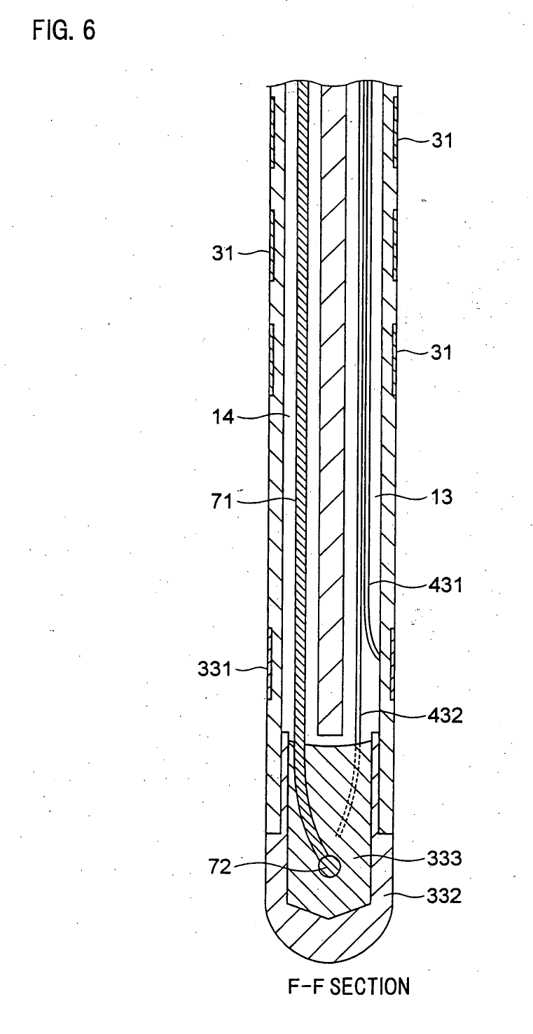

FIG. 7 is a longitudinal sectional view (illustrating a section G-G in FIG. 3) in the distal region of the multi-lumen tube.

FIG. 8 is a longitudinal sectional view (illustrating a section I-I in FIG. 3) in an intermediate region of the multi-lumen tube.

FIG. 9 is an explanatory view typically illustrating the interior of a handle making up the intracardiac defibrillation catheter according to the present invention.

FIG. 10 is an explanatory view illustrating, on an enlarged scale, a proximal end portion of the multi-lumen tube connected to the handle.

FIG. 11 is a potential waveform diagram measured when predetermined electric energy has been applied by the intracardiac defibrillation catheter according to the present invention.

FIG. 12 is cross-sectional views illustrating modified examples of the intracardiac defibrillation catheter according to the present invention.

### MODE FOR CARRYING OUT THE INVENTION

An embodiment of the intracardiac defibrillation catheter according to the present invention will hereinafter be described.

The intracardiac defibrillation catheter 100 according to this embodiment is equipped with a multi-lumen tube 10, a handle 20, a first DC electrode group 31G, a second DC electrode group 32G, a distal-side potential-measuring electrode group 33G, a proximal-side potential-measuring electrode group 34G, a first lead wire group 41G, a second lead wire group 42G, a third lead wire group 43G, a fourth lead wire group 44G, a first external cord 51, a second external cord 52, a third external cord 53, a first connector 61, a second connector 62 and a third connector 63.

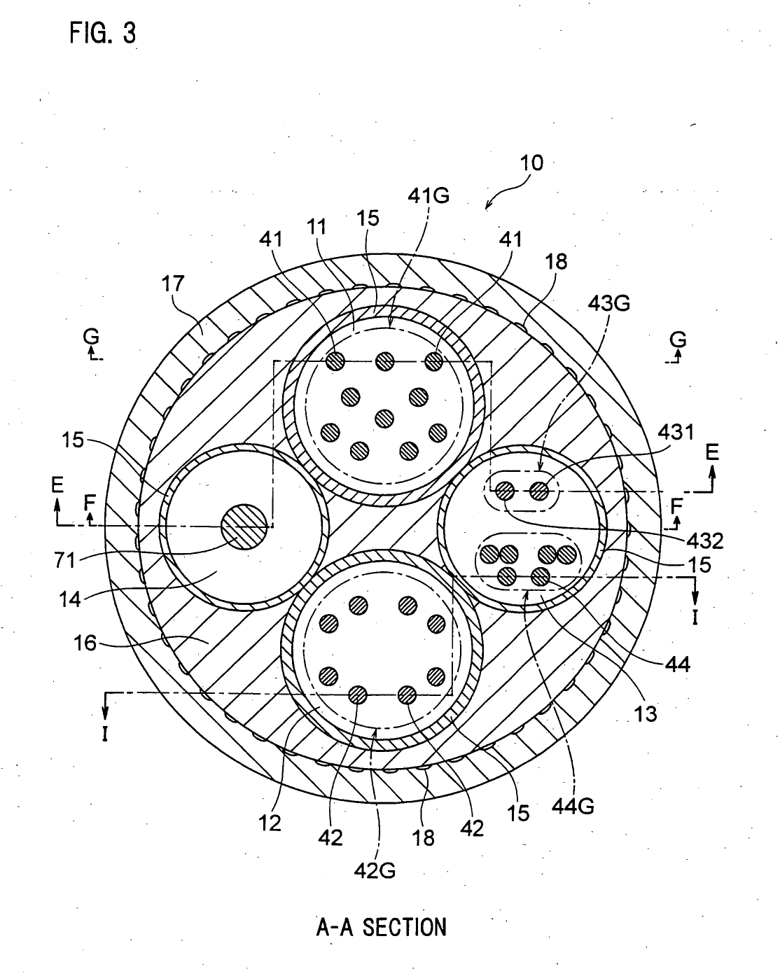

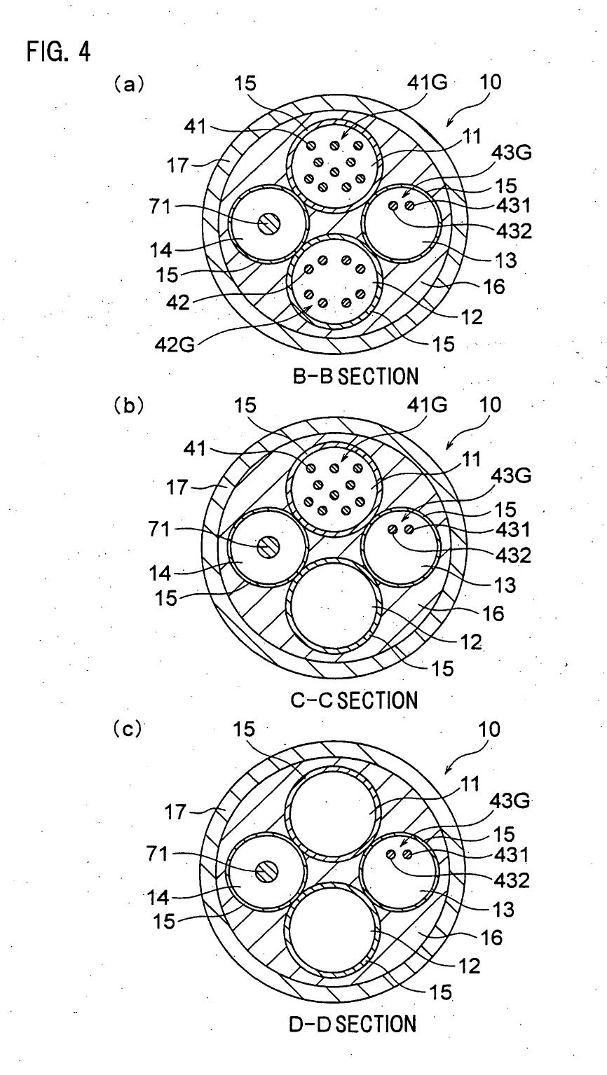

As illustrated in FIGS. 3 and 4, four lumens (a first lumen 11, a second lumen 12, a third lumen 13 and a fourth lumen 14) are formed in the multi-lumen tube 10 (an insulated tube member having a multi-lumen structure) making up the intracardiac defibrillation catheter 100 according to this embodiment.

In FIGS. 3 and 4, reference numerals 15, 16 and 17 designate a fluororesin layer partitioning into the lumens, an inner (core) part composed of a nylon elastomer having a low hardness and an outer (shell) part composed of a nylon elastomer having a high hardness, respectively. In FIG. 3, reference numeral 18 designates a stainless steel wire forming a braid.

The fluororesin layer 15 partitioning into the lumens is formed of a material having high insulating property, for example, a perfluoroalkyl vinyl ether copolymer (PFA) or polytetrafluoroethylene (PTFE).

The nylon elastomer forming the outer part 17 of the multi-lumen tube 10, having different hardnesses in an axial direction, is used, whereby the multi-lumen tube 10 is formed in such a manner that the hardness becomes higher stepwise toward the proximal side from the distal side.

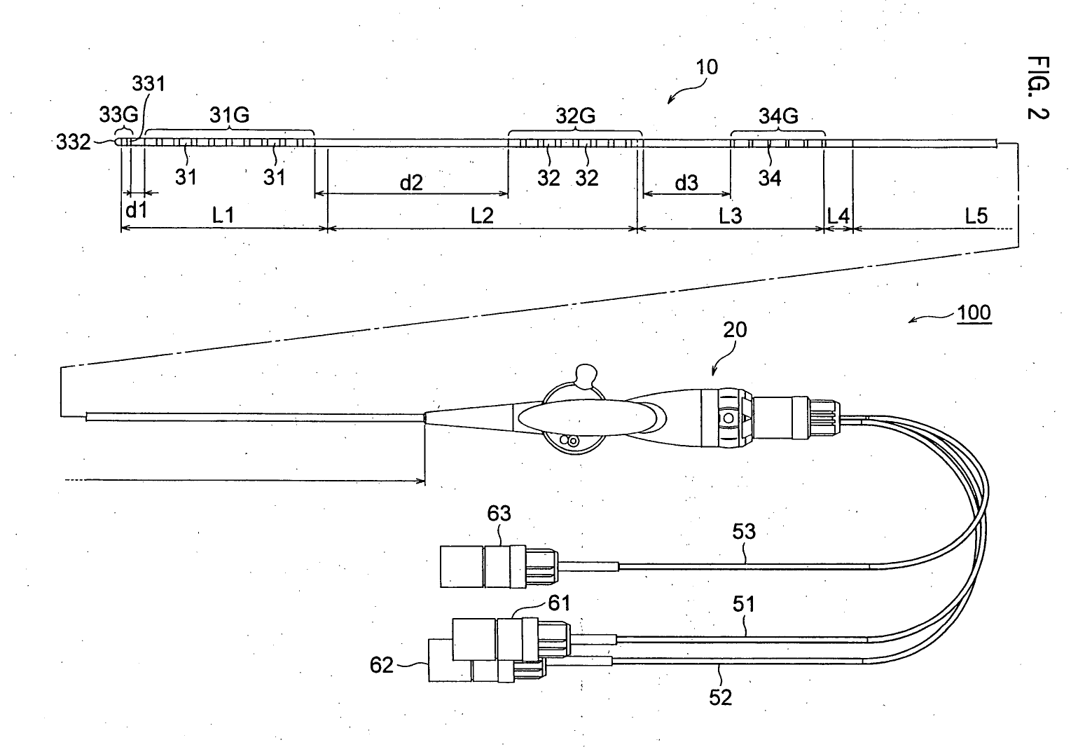

As a preferable example, the hardness (hardness as measured by a D-type hardness meter) in a region indicated by L1 (length: 65 mm) in FIG. 2 is 40, the hardness in a region indicated by L2 (length: 110 mm) is 55, the hardness in a region indicated by L3 (length: 60 mm) is 63, the hardness in a region indicated by L4 (length: 10 mm) is 68, and the hardness in a region indicated by L5 (length: 500 mm) is 72.

The braid formed by the stainless steel wire 18 is formed in only the region indicated by L5 in FIG. 2 and is provided between the inner part 16 and the outer part 17 as illustrated in FIG. 3.

The outer diameter of the multi-lumen tube 10 is, for example, 1.2 to 3.3 mm.

No particular limitation is imposed on a method for producing the multi-lumen tube 10.

The handle 20 making up the intracardiac defibrillation catheter 100 according to this embodiment is equipped with a handle body 21, a lug 22, a connector part 23 and a strain relief 24.

The lug 22 is rotationally operated, whereby a distal end portion of the multi-lumen tube 10 can be deflected (oscillated).

The first DC electrode group 31G, the second DC electrode group 32G, the distal-side potential-measuring electrode group 33G and the proximal-side potential-measuring electrode group 34G are installed on an outer periphery (a distal region where no braid is formed in the interior thereof) of the multi-lumen tube 10. Here, “the electrode group” means an assembly of a plurality of electrodes that are installed at narrow intervals (for example, 5 mm or less), and form the same pole (have the same polarity) or have the same object.

The first DC electrode group is composed of a plurality of electrodes forming the same pole (minus pole or plus pole) and installed at narrow intervals in the distal region of the multi-lumen tube. Here, the number of the electrodes making up the first DC electrode group is, for example, 8 to 12, preferably 8 to 10 though it varies according to the width of individual electrodes and arrangement interval.

In this embodiment, the first DC electrode group 31G is made up of 10 ring-like electrodes 31 installed in the distal region of the multi-lumen tube 10.

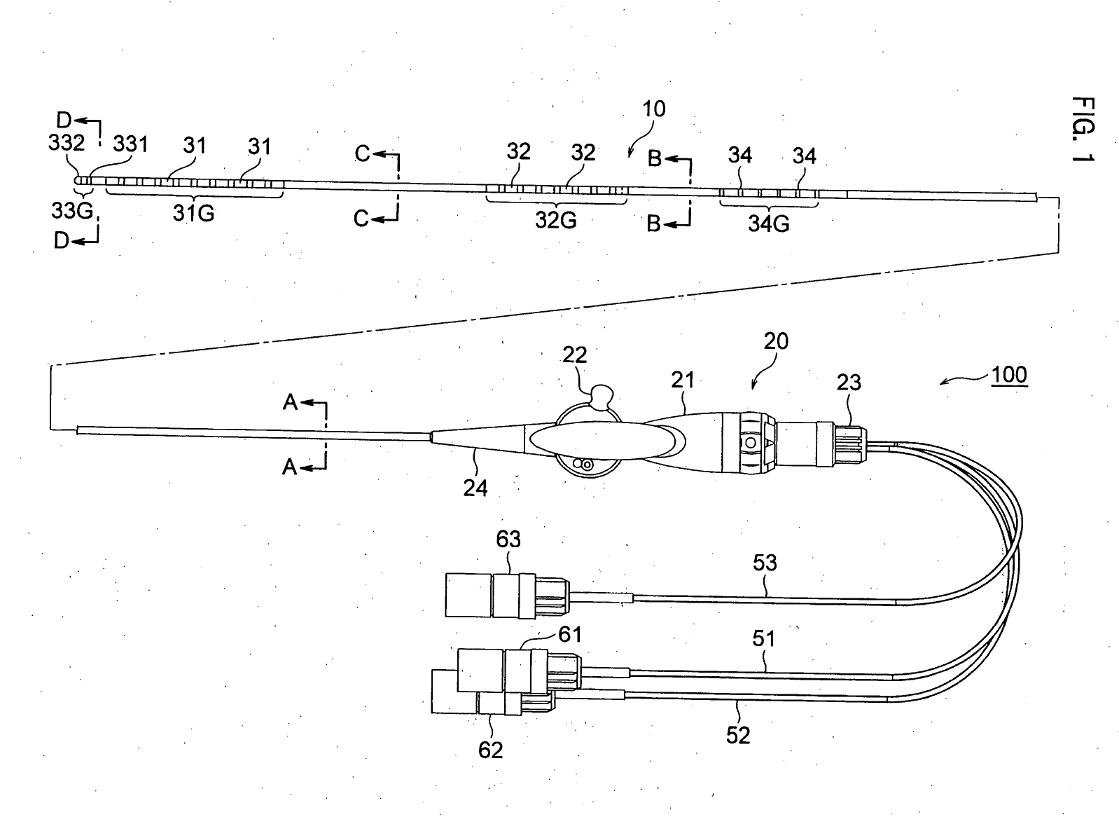

The electrodes 31 making up the first DC electrode group 31G are connected to terminals having the same pole in a direct current power unit through lead wires (lead wires 41 making up the first lead wire group 41G) and the connector (first connector 61 illustrated in FIG. 1).

Here, the width (length in an axial direction) of the electrode 31 is preferably 3 to 5 mm, and is 4 mm as a preferable example.

If the width of the electrode 31 is too narrow, the quantity of heat generated upon application of voltage becomes excessive, and so there is a possibility that a surrounding tissue may be damaged. If the width of the electrode 31 is too wide on the other hand, the flexibility or softness of a portion of the multi-lumen tube 10, in which the first DC electrode group 31G is provided, may be impaired in some cases.

An installation interval (clearance distance between adjoining electrodes) between the electrodes 31 is preferably 1 to 3 mm, and is 2 mm as a preferable example.

The first DC electrode group 31G is located in, for example, a coronary vein upon use (upon arrangement into a cardiac cavity) of the intracardiac defibrillation catheter 100.

The second DC electrode group is composed of a plurality of electrodes forming a pole (plus pole or minus pole) opposite to the first DC electrode group and installed at narrow intervals on the multi-lumen tube towards proximal direction from the installation position of the first DC electrode group. Here, the number of the electrodes making up the second DC electrode group is, for example, 6 to 10, preferably 8 to 10 though it varies according to the width of individual electrodes and arrangement interval.

In this embodiment, the second DC electrode group 32G is made up of 8 ring-like electrodes 32 installed on the multi-lumen tube 10 towards proximal direction from the installation position of the first DC electrode group 31G. The electrodes 32 making up the second DC electrode group 32G are connected to terminals (terminals having a pole opposite to those, to which the first DC electrode group 31G is connected) having the same pole in the direct current power unit through lead wires (lead wires 42 making up the second lead wire group 42G) and the connector (second connector 62 illustrated in FIG. 1).

Voltages different in polarity from each other are thereby respectively applied to the first DC electrode group 31G (electrodes 31) and the second DC electrode group 32G (electrodes 32), and so the first DC electrode group 31G and the second DC electrode group 32G become electrode groups different in polarity from each other (when the polarity of one electrode group is minus, the polarity of the other electrode group becomes plus).

Here, the width (length in an axial direction) of the electrode 32 is preferably 3 to 5 mm, and is 4 mm as a preferable example.

If the width of the electrode 32 is too narrow, the quantity of heat generated upon application of voltage becomes excessive, and so there is a possibility that a surrounding tissue may be damaged. If the width of the electrode 32 is too wide on the other hand, the flexibility or softness of a portion of the multi-lumen tube 10, in which the second DC electrode group 32G is provided, may be impaired in some cases.

An installation interval (clearance distance between adjoining electrodes) between the electrodes 32 is preferably 1 to 3 mm, and is 2 mm as a preferable example.

The second DC electrode group 32G is located in, for example, a right atrium upon use (upon arrangement into a cardiac cavity) of the intracardiac defibrillation catheter 100.

In this embodiment, the distal-side potential-measuring electrode group 33G is made up of a ring-like electrode 331 installed on the multi-lumen tube 10 towards distal direction from the installation position of the first DC electrode group 31G, and a distal-end tip electrode 332.

The electrodes 331 and 332 making up the distal-side potential-measuring electrode group 33G are connected to an electrocardiograph through lead wires (lead wire 431 and lead wire 432 making up the third lead wire group 43G) and the connector (third connector 63 illustrated in FIG. 1). The electrode 331 is thereby clearly distinguished from the electrodes 31 connected to the direct current power unit.

Here, the width (length in an axial direction) of the electrode 331 is preferably 0.5 to 2.0 mm, and is 1.2 mm as a preferable example. The width of the electrode 332 is preferably 1.0 to 4.0 mm, and is 2 mm as a preferable example.

If the widths of the electrodes 331 and 332 are too wide, the measurement accuracy of a cardiac potential is lowered, and it is difficult to ascertain a site where an abnormal potential has been generated.

An installation interval (clearance distance) between the electrodes 331 and 332 is preferably 1.0 to 2.5 mm, and is 2 mm as a preferable example.

In this embodiment, the proximal-side potential-measuring electrode group 34G is made up of 6 ring-like electrodes 34 installed on the multi-lumen tube 10 towards proximal direction from the installation position of the second DC electrode group 32G.

The electrodes 34 making up the proximal-side potential-measuring electrode group 34G are connected to the electrocardiograph through lead wires (lead wires 44 making up the fourth lead wire group 44G) and the connector (third connector 63 illustrated in FIG. 1).

Here, the width (length in an axial direction) of the electrode 34 is preferably 0.5 to 2.0 mm, and is 1.2 mm as a preferable example.

If the width of the electrode 34 is too wide, the measurement accuracy of a cardiac potential is lowered, and it is difficult to ascertain a site where an abnormal potential has been generated.

An installation interval (clearance distance between adjoining electrodes) between the electrodes 34 is preferably 1.0 to 10.0 mm, and is 5 mm as a preferable example.

The proximal-side potential-measuring electrode group 34G is located in, for example, a superior vena cava where an abnormal potential tend to generate upon use (upon arrangement into a cardiac cavity) of the intracardiac defibrillation catheter 100.

A clearance distance d1 between the distal-side potential-measuring electrode group 33G (electrode 331) and the first DC electrode group 31G (distal-side electrode 31) is preferably 0.5 to 20 mm, and is 5 mm as a preferable example.

A clearance distance d2 between the first DC electrode group 31G (proximal-side electrode 31) and the second DC electrode group 32G (distal-side electrode 32) is preferably 40 to 100 mm, and is 66 mm as a preferable example.

A clearance distance d3 between the second DC electrode group 32G (proximal-side electrode 32) and the proximal-side potential-measuring electrode group 34G (distal-side electrode 34) is preferably 5 to 50 mm, and is 30 mm as a preferable example.

The electrodes 31, 32, 331, 332 and 34 making up the first DC electrode group 31G, the second DC electrode group 32G, the distal-side potential-measuring electrode group 33G and the proximal-side potential-measuring electrode group 34G are preferably formed of platinum or a platinum-based alloy for the purpose of making radiopacity good.

The first lead wire group 41G illustrated in FIGS. 3 and 4 is an assembly of 10 lead wires 41 respectively connected to the 10 electrodes (31) making up the first DC electrode group (31G).

Each of the 10 electrodes 31 making up the first DC electrode group 31G can be electrically connected to the direct current power unit through the first lead wire group 41G (lead wire 41).

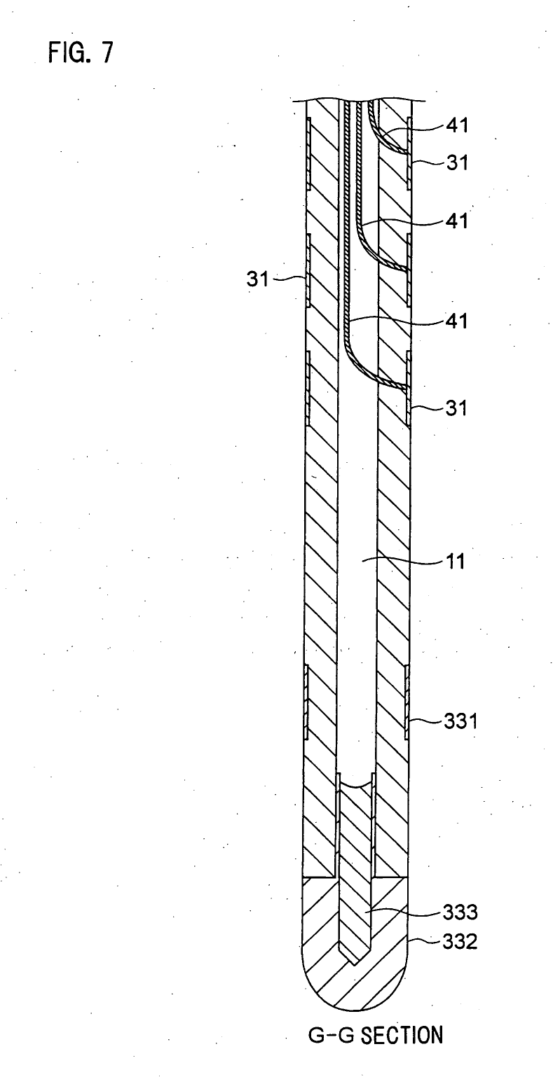

As illustrated in FIG. 7, the electrodes 31 (3 electrodes among the 10 electrodes are illustrated in FIG. 7) making up the first DC electrode group 31G are respectively connected to the separate lead wires 41. Each of the lead wires 41 is welded at its distal end portion to an inner peripheral surface of the electrode 31 and enters the first lumen 11 from a side hole (not illustrated) formed in a tube wall of the multi-lumen tube 10. The 10 lead wires 41 entered into the first lumen 11 extend into the first lumen 11 as the first lead wire group 41G.

The second lead wire group 42G illustrated in FIGS. 3 and 4 is an assembly of 8 lead wires 42 respectively connected to the 8 electrodes (32) making up the second DC electrode group (32G).

Each of the 8 electrodes 32 making up the second DC electrode group 32G can be electrically connected to the direct current power unit through the second lead wire group 42G (lead wire 42).

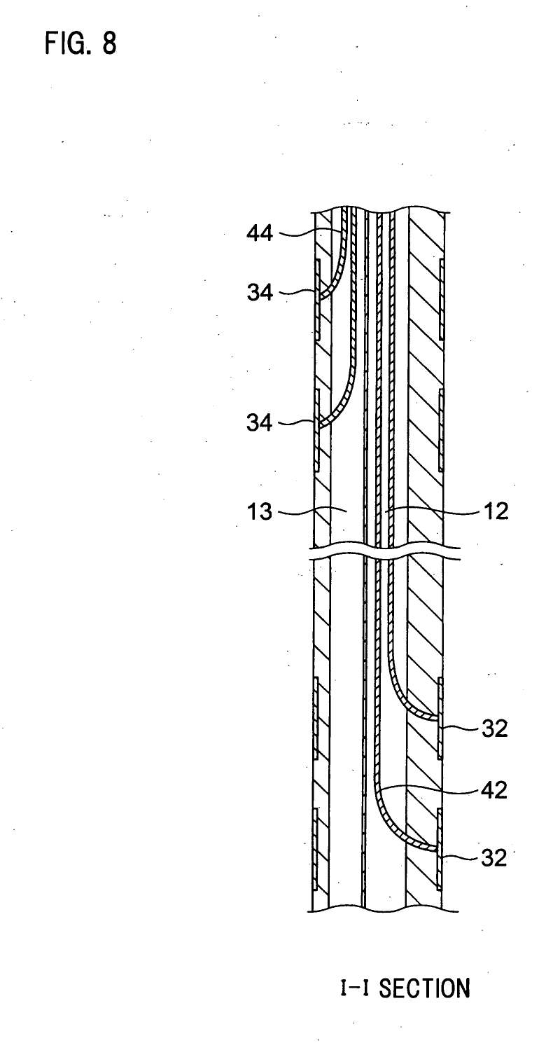

As illustrated in FIG. 8, the electrodes 32 (2 electrodes among the 8 electrodes are illustrated in FIG. 8) making up the second DC electrode group 32G are respectively connected to the separate lead wires 42. Each of the lead wires 42 is welded at its distal end portion to an inner peripheral surface of the electrode 32 and enters the second lumen 12 (lumen different from the first lumen 11 to which the first lead wire group 41G extends) from a side hole (not illustrated) formed in the tube wall of the multi-lumen tube 10. The 8 lead wires 42 entered into the second lumen 12 extend into the second lumen 12 as the second lead wire group 42G.

The first lead wire group 41G extends into the first lumen 11, and the second lead wire group 42G extends into the second lumen 12 as described above, whereby both lead wire groups are completely insulated and isolated from each other within the multi-lumen tube 10. Thus, short circuit between the first lead wire group 41G (first DC electrode group 31G) and the second lead wire group 42G (second DC electrode group 32G) can be surely prevented when the voltage necessary for the defibrillation is applied.

The third lead wire group 43G illustrated in FIGS. 3 and 4 is an assembly of the lead wire 431 connected to the ring-like electrode (331) and the lead wire 432 connected to the distal-end tip electrode (332), which make up the distal-side potential-measuring electrode group (33G).

The electrodes 331 and 332 making up the distal-side potential-measuring electrode group 33G can be respectively connected to the electrocardiograph through the third lead wire group 43G (lead wires 431 and 432).

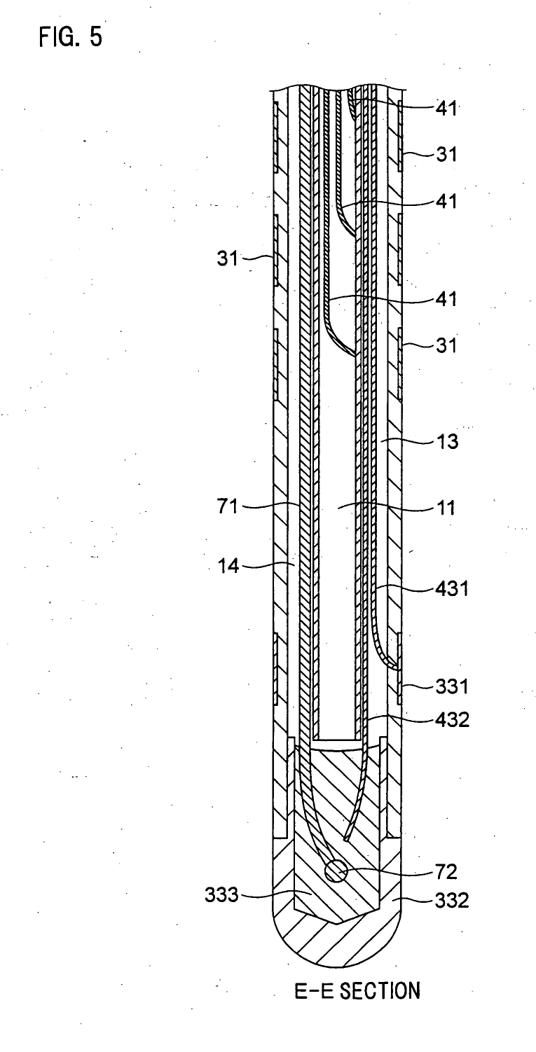

As illustrated in FIG. 5, the lead wire 431 is welded at its distal end portion to an inner peripheral surface of the electrode 331 and enters the third lumen 13 (lumen different from the first lumen 11 to which the first lead wire group 41G extends and the second lumen 12 to which the second lead wire group 42G extends) from a side hole (not illustrated) formed in the tube wall of the multi-lumen tube 10. The lead wire 432 is bonded at its distal end to the distal-end tip electrode 332 with solder 333 and enters the third lumen 13. The lead wires 431 and 432 entered into the third lumen 13 extend into the third lumen 13 as the third lead wire group 43G.

The third lead wire group 43G extending into the third lumen 13 as described above is completely insulated and isolated from both first lead wire group 41G and second lead wire group 42G. Thus, short circuit between the third lead wire group 43G (distal-side potential-measuring electrode group 33G) and the first lead wire group 41G (first DC electrode group 31G) or the second lead wire group 42G (second DC electrode group 32G) can be surely prevented when the voltage necessary for the defibrillation is applied.

The fourth lead wire group 44G illustrated in FIG. 3 is an assembly of 6 lead wires 44 respectively connected to the electrodes (34) making up the proximal-side potential-measuring electrode group (34G).

Each of the electrodes 34 making up the proximal-side potential-measuring electrode group 34G can be connected to the electrocardiograph through the fourth lead wire group 44G (lead wire 44).

As illustrated in FIG. 8, the electrodes 34 (2 electrodes among the 6 electrodes are illustrated in FIG. 8) making up the proximal-side potential-measuring electrode group 34G are respectively connected to the separate lead wires 44. Each of the lead wires 44 is welded at its distal end portion to an inner peripheral surface of the electrode 34 and enters the third lumen 13 from a side hole (not illustrated) formed in the tube wall of the multi-lumen tube 10. The 6 lead wires 44 entered into the third lumen 13 extend into the third lumen 13 as the fourth lead wire group 44G.

The fourth lead wire group 44G extending into the third lumen 13 as described above is completely insulated and isolated from both first lead wire group 41G and second lead wire group 42G. Thus, short circuit between the fourth lead wire group 44G (proximal-side potential-measuring electrode group 34G) and the first lead wire group 41G (first DC electrode group 31G) or the second lead wire group 42G (second DC electrode group 32G) can be surely prevented when the voltage necessary for the defibrillation is applied.

The lead wires 41, the lead wires 42, the lead wire 431, the lead wire 432 and the lead wires 44 are each composed of a resin-coated wire obtained by coating an outer periphery of a metal conductor with a resin such as polyimide. The coating thickness of the resin is controlled to about 5 to 10 μm.

In FIGS. 3 and 4, reference numeral 71 designates a pull wire.

The pull wire 71 extends into the fourth lumen 14 and located eccentrically from a central axis of the multi-lumen tube 10.

As illustrated in FIG. 5, a distal end portion of the pull wire 71 is fixed to the distal-end tip electrode 332 with solder 333. A large-diameter portion (retaining portion) 72 for fall stopping is formed at the tip of the pull wire 71. The distal-end tip electrode 332 can be thereby firmly bonded to the pull wire 71 to surely prevent the distal-end tip electrode 332 from falling.

On the other hand, a proximal end portion of the pull wire 71 is connected to the lug 22 of the handle 20, and the pull wire 71 is pulled by operating the lug 22, whereby a distal end portion of the multi-lumen tube 10 is deflected.

The pull wire 71 is formed by a stainless steel or Ni—Ti-based super-elastic alloy. However, the pull wire may not necessarily formed by a metal. The pull wire 71 may be formed by, for example, a nonconductive wire having high strength.

Incidentally, the mechanism for deflecting the distal end portion of the multi-lumen tube is not limited to this, and it may be constructed by providing, for example, a plate spring.

Only the pull wire 71 extends into the fourth lumen 14 of the multi-lumen tube 10, and no lead wire (group) extends. The lead wires can thereby be prevented from being damaged (for example, abraded) by the pull wire 71 moving in the axial direction upon the distal end deflection operation of the multi-lumen tube 10.

In the intracardiac defibrillation catheter 100 according to this embodiment, the first lead wire group 41G, the second lead wire group 42G, and the third lead wire group 43G and the fourth lead wire group G are insulated and isolated from one another even in the interior of the handle 20.

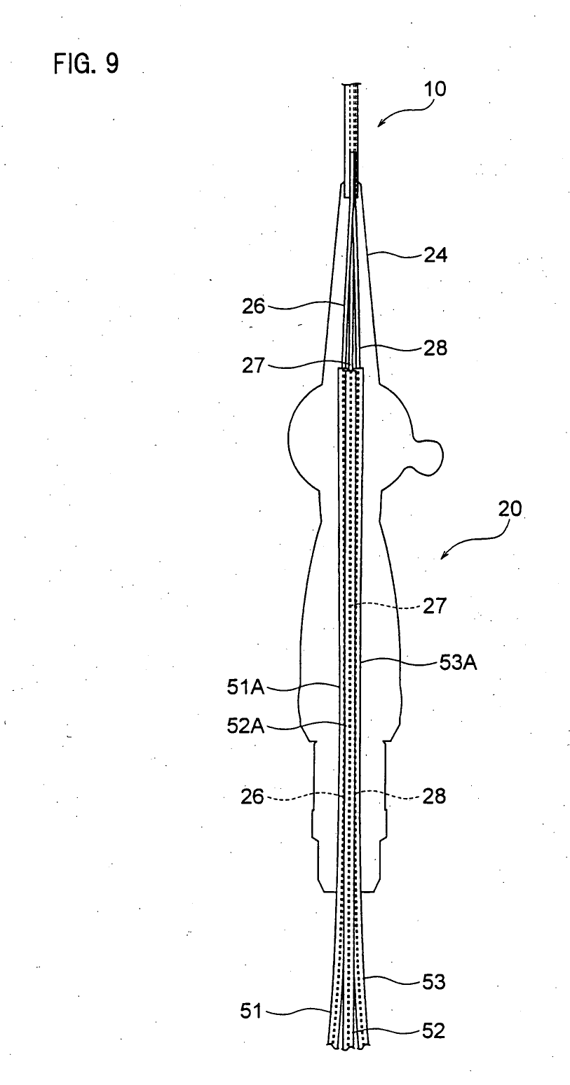

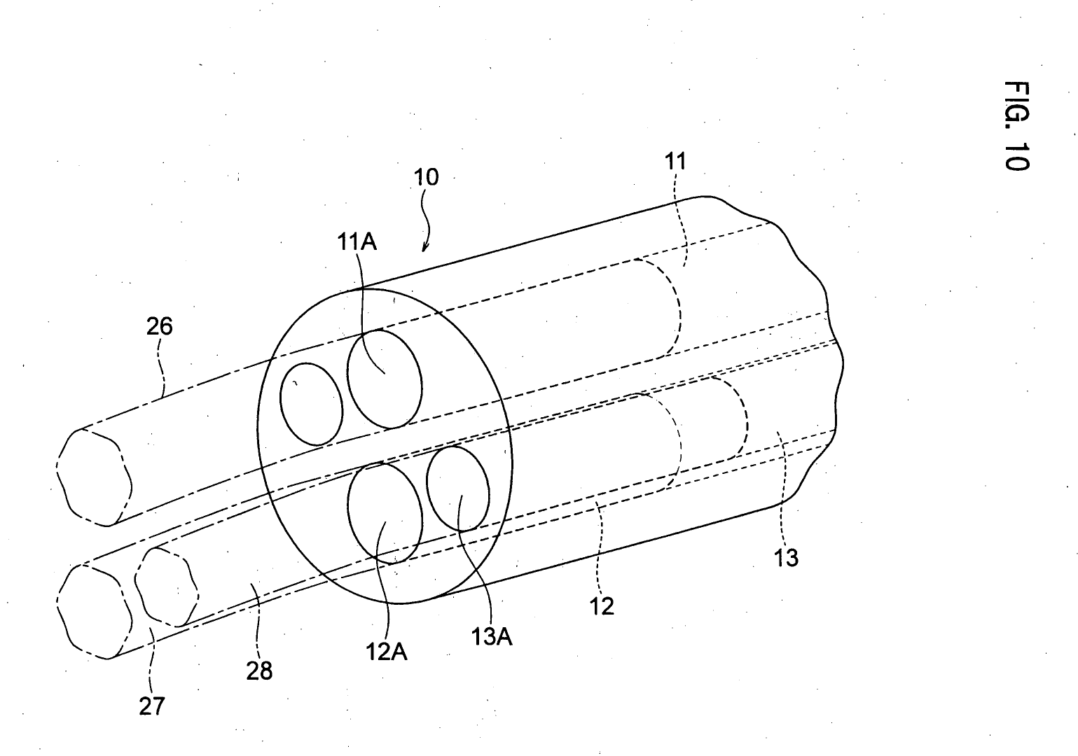

FIG. 9 is an explanatory view typically illustrating the interior of the handle 20 making up the intracardiac defibrillation catheter 100, and FIG. 10 is an explanatory view illustrating, on an enlarged scale, a proximal end portion of the multi-lumen tube 10 connected to the handle 20. Incidentally, the lead wire groups and the pull wire are omitted from illustration in FIGS. 9 and 10.

As illustrated in FIG. 9, the proximal end portion of the multi-lumen tube 10 is inserted into a distal end opening of the handle 20 (strain relief 24), whereby the multi-lumen tube 10 is connected to the handle 20.

In the interior of the handle 20, 3 insulated tubes (first insulated tube 26, second insulated tube 27 and third insulated tube 28), into which the respective lead wire groups are inserted, extend.

As illustrated in FIG. 10, a distal end portion (portion about 10 mm long from the tip) of the first insulated tube 26 is inserted into the first lumen 11 from an opening 11A, whereby the first insulated tube 26 is connected to the first lumen 11 into which the first lead wire group extends.

The first insulated tube 26 connected to the first lumen 11 passes through an inner hole of a first protecting tube 51A extending in the interior of the handle 20 and extends to a connector (not illustrated, the first connector 61 illustrated in FIG. 1) located outside the handle 20 to form a passage through which the proximal end portion of the first lead wire group is guided to the connector.

The first lead wire group extended out from the multi-lumen tube 10 thereby extends into the first insulated tube 26 and is connected to the connector (first connector 61).

Incidentally, the first protecting tube 51A, into which the first insulated tube 26 is inserted, extends from the handle 20 to the outside to form an external cord (first external cord 51 illustrated in FIG. 1).

As illustrated in FIG. 10, a distal end portion (portion about 10 mm long from the tip) of the second insulated tube 27 is inserted into the second lumen 12 from an opening 12A, whereby the second insulated tube 27 is connected to the second lumen 12 into which the second lead wire group extends.

The second insulated tube 27 connected to the second lumen 12 passes through an inner hole of a second protecting tube 52A extending in the interior of the handle 20 and extends to a connector (not illustrated, the second connector 62 illustrated in FIG. 1) located outside the handle 20 to form a passage through which the proximal end portion of the second lead wire group is guided to the connector.

The second lead wire group extended out from the multi-lumen tube 10 thereby extends into the second insulated tube 27 and is connected to the connector (second connector 62).

Incidentally, the second protecting tube 52A, into which the second insulated tube 27 is inserted, extends from the handle 20 to the outside to form an external cord (second external cord 52 illustrated in FIG. 1).

As illustrated in FIG. 10, a distal end portion (portion about 10 mm long from the tip) of the third insulated tube 28 is inserted into the third lumen 13 from an opening 13A, whereby the third insulated tube 28 is connected to the third lumen 13 into which the third lead wire group and the fourth lead wire group extend.

The third insulated tube 28 connected to the third lumen 13 passes through an inner hole of a third protecting tube 53A extending in the interior of the handle 20 and extends to a connector (not illustrated, the third connector 63 illustrated in FIG. 1) located outside the handle 20 to form a passage through which the proximal end portions of the third lead wire group and fourth lead wire group are guided to the connector.

The third lead wire group and fourth lead wire group extended out from the multi-lumen tube 10 thereby extend into the third insulated tube 28 and is connected to the connector (third connector 63).

Incidentally, the third protecting tube 53A, into which the third insulated tube 28 is inserted, extends out of the handle 20 to form an external cord (third external cord 53 illustrated in FIG. 1).

As examples of materials forming the insulated tubes (first insulated tube 26, second insulated tube 27 and third insulated tube 28), may be mentioned polyimide resins, polyamide resins and polyamide-imide resins. The wall thickness of each insulated tube is preferably 20 to 40 μm, and is 30 μM as a preferable example.

As examples of materials forming the protecting tubes (first protecting tube 51A, second protecting tube 52A and third protecting tube 53A), into which the insulated tubes are respectively inserted, may be mentioned nylon-based elastomers such as “Pebax” (product of ARKEMA CO.).

According to the intracardiac defibrillation catheter 100 of this embodiment having such a structure as described above, the first lead wire group 41G extends into the first insulated tube 26, the second lead wire group 42G extends into the second insulated tube 27, and the third lead wire group 43G and the fourth lead wire group 44G extend into the third insulated tube 28, whereby the first lead wire group 41G, the second lead wire group 42G, and the third lead wire group 43G and the fourth lead wire group 44G can be completely insulated and isolated from one another even in the interior of the handle 20. As a result, short circuit (in particular, short circuit between lead wire groups extended out in the vicinity of the openings of the lumens) between the first lead wire group 41G, the second lead wire group 42G and the third lead wire group 43G or the fourth lead wire group 44G in the interior of the handle 20 can be surely prevented when the voltage necessary for the defibrillation is applied.

In addition, the insulated tubes (first insulated tube 26, second insulated tube 27 and third insulated tube 28) are respectively protected by the protecting tubes (first protecting tube 51A, second protecting tube 52A and third protecting tube 53A), whereby the insulated tubes can be prevented from being damaged by, for example, contact or abrasion with a member of the lug 22 upon a distal end deflection operation of the multi-lumen tube 10.

In the intracardiac defibrillation catheter 100 according to this embodiment, each of the 10 electrodes 31 making up the first DC electrode group 31G is connected to a terminal of one pole in the direct current power unit through each of the 10 lead wires 41 making up the first lead wire group 41G and the first connector 61.

On the other hand, each of the 8 electrodes 32 making up the second DC electrode group 32G is connected to a terminal of the other pole in the direct current power unit through each of the 8 lead wires 42 making up the second lead wire group 42G and the second connector 62.

Further, the 2 electrodes (electrode 331 and electrode 332) making up the distal-side potential-measuring electrode group 33G are connected to the electrocardiograph through the 2 lead wires (lead wire 431 and lead wire 432) making up the third lead wire group 43G and the third connector 63.

Furthermore, the 6 electrodes 34 making up the proximal-side potential-measuring electrode group 34G are connected to the electrocardiograph through the 6 lead wires 44 making up the fourth lead wire group 44G and the third connector 63.

The intracardiac defibrillation catheter 100 according to this embodiment is a catheter used for applying a direct current voltage between the first DC electrode group 31G and the second DC electrode group 32G, thereby directly applying electric energy to a heart that has suffered fibrillation to conduct a defibrillation treatment, and is different from the conventionally known electrode catheter used in diagnosis of arrhythmia (measurement of cardiac potential) or a cauterization treatment in use and function.

The intracardiac defibrillation catheter 100 according to this embodiment is suitably used upon cardiac catheterization during which atrial fibrillation is liable to occur. Particularly preferably, the intracardiac defibrillation catheter 100 is inserted into a cardiac cavity of a patient in advance to conduct the cardiac catheterization.

The intracardiac defibrillation catheter 100 is inserted into a cardiac cavity in such a manner that the first DC electrode group 31G is located in a coronary vein, and the second DC electrode group 32G is located in a right atrium, thereby creating a state that the heart is held between the first DC electrode group 31G and the second DC electrode group 32G.

An electrocardiogram measured by the distal-side potential-measuring electrode group 33G or the proximal-side potential-measuring electrode group 34G during cardiac catheterization is monitored to stop the cardiac catheterization when atrial fibrillation has occurred so as to conduct a defibrillation treatment by the intracardiac defibrillation catheter 100. Specifically, a direct current voltage is applied between the first DC electrode group 31G and the second DC electrode group 32G through the first lead wire group 41G and the second lead wire group 42G to directly give electric energy to the heart that has suffered the fibrillation.

Here, the electric energy supplied to the heart by the intracardiac defibrillation catheter 100 is preferably 10 to 30 J.

If the electric energy is too small, it is impossible to sufficiently conduct the defibrillation treatment. If the electric energy is excessive on the other hand, tissues about the first DC electrode group 31G and the second DC electrode group 32G may possibly be damaged.

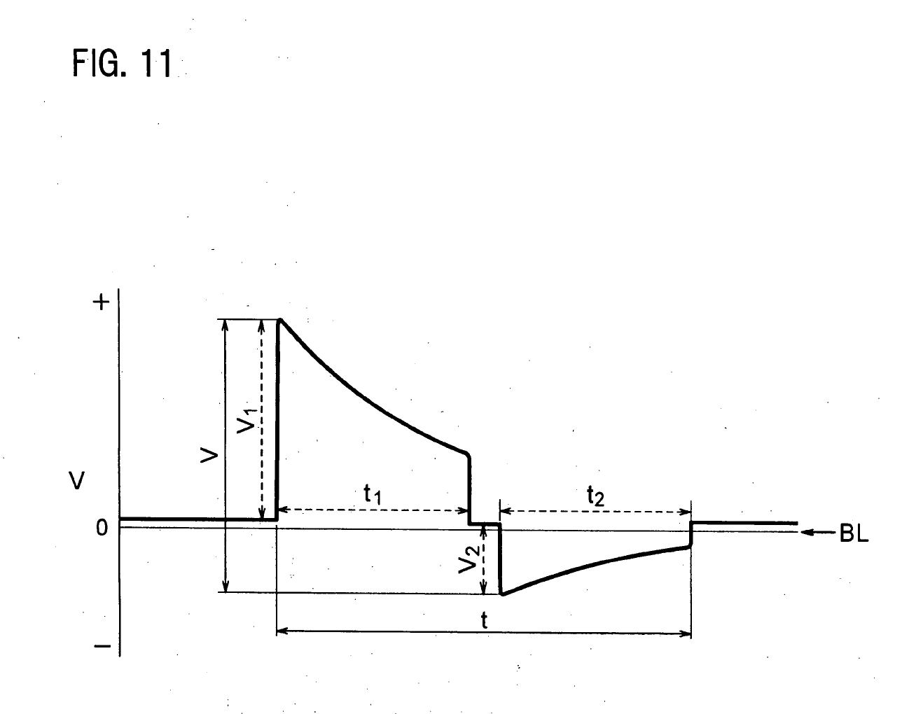

FIG. 11 is a potential waveform diagram measured when predetermined electric energy (for example, setting output=10 J) has been applied by the intracardiac defibrillation catheter 100 according to this embodiment. In this diagram, an axis of abscissa and an axis of ordinate indicate a time and a potential, respectively.

First, a direct current voltage is applied between the first DC electrode group 31G and the second DC electrode group 32G in such a manner that the first DC electrode group 31G becomes a minus pole and the second DC electrode group 32G becomes a plus pole, whereby electric energy is supplied to build up a measuring potential (V₁ is a peak voltage at this time). After a fixed time (t₁) has elapsed, a direct current voltage that the poles have been reversed in such a manner that the first DC electrode group 31G becomes a plus pole and the second DC electrode group 32G becomes a minus pole is applied between both electrode groups, whereby electric energy is supplied to build up a measuring potential (V₂ is a peak voltage at this time).

Here, the time (t₁) is, for example, 1.5 to 10.0 seconds, and the peak voltage (V₁) measured is, for example, 300 to 500V.

In the intracardiac defibrillation catheter 100 according to this embodiment, high electric energy is supplied (high voltage is applied) though it is low compared with AED, so that it is necessary to surely prevent short circuit, which has not become a problem in the conventional electrode catheter, to ensure safety.

Thus, in the intracardiac defibrillation catheter 100, the first lead wire group 41G connected to the first DC electrode group 31G is caused to extend into the first lumen 11 formed in the multi-lumen tube 10 and into the first insulated tube 26 in the interior of the handle 20 to be connected to the first connector 61, the second lead wire group 42G connected to the second DC electrode group 32G is caused to extend into the second lumen 12 formed in the multi-lumen tube 10 and into the second insulated tube 27 in the interior of the handle 20 to be connected to the second connector 62, and the third lead wire group 43G connected to the distal-side potential-measuring electrode group 33G and the fourth lead wire group 44G connected to the proximal-side potential-measuring electrode group 34G are respectively caused to extend into the third lumen 13 formed in the multi-lumen tube 10 and into the third insulated tube 28 in the interior of the handle 20 to be connected to the third connector 63.

The first lead wire group 41G, the second lead wire group 42G, and the third lead wire group 43G and the fourth lead wire group 44G can thereby be completely insulated and isolated from one another in the interior of the multi-lumen tube 10 and the interior of the handle 20.

Accordingly, short circuit among the first lead wire group 41G (first DC electrode group 31G), the second lead wire group 42G (second DC electrode group 32G) and the third lead wire group 43G or the fourth lead wire group 44G (distal-side potential-measuring electrode group 33G or proximal-side potential-measuring electrode group 34G) can be surely prevented when the voltage necessary for the defibrillation is applied.

One embodiment of the present invention has been described above. However, the intracardiac defibrillation catheter according to the present invention is not limited thereto, and various modifications may be made.

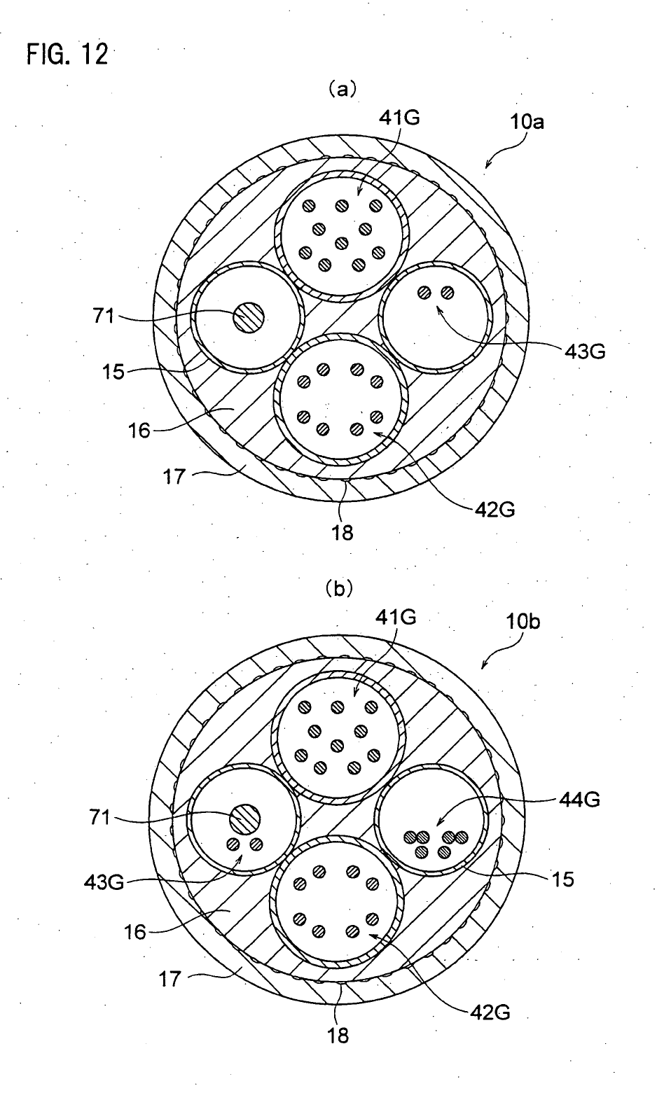

For example, only the distal-side potential-measuring electrode group 33G may be provided as a potential-measuring electrode group, and only the third lead wire group 43G may extend into the third lumen 13 as a lead wire group for potential measurement as illustrated in FIG. 12(a).

Alternatively, the third lead wire group 43G may extend into the fourth lumen 14 together with the pull wire 71, and the fourth lead wire group 44G may extend into the third lumen 13 as illustrated in FIG. 12(b).

Further, only the proximal-side potential-measuring electrode group may be provided as a potential-measuring electrode group, and only the fourth lead wire group may extend into the third lumen 13 as a lead wire group for potential measurement.

### DESCRIPTION OF CHARACTERS

100 Intracardiac defibrillation catheter
10 Multi-lumen tube
11 First lumen
12 Second lumen
13 Third lumen
14 Fourth lumen
15 Fluororesin layer
16 Inner (core) part
17 Outer (shell) part
18 Stainless steel wire
20 Handle
21 Handle body
22 Lug
23 Connector part
24 Strain relief
26 First insulated tube
27 Second insulated tube
28 Third insulated tube
31G First DC electrode group
31 Ring-like electrodes
32G Second DC electrode group
32 Ring-like electrodes
33G Distal-side potential-measuring electrode group
331 Ring-like electrode
332 Distal end tip electrode
333 Solder
34G Proximal-side potential-measuring electrode group
34 Ring-like electrodes
41G First lead wire group
41 Lead wires
42G Second lead wire group
42 Lead wires
43G Third lead wire group
431 Lead wire
432 Lead wire
44G Fourth lead wire group
44 Lead wires
51 First external cord
52 Second external cord
53 Third external cord
51A First protecting tube
52A Second protecting tube
53A Third protecting tube
61 First connector
62 Second connector
63 Third connector
71 Pull wire
72 Large-diameter portion for fall stopping

1. An intracardiac defibrillation catheter inserted into a cardiac cavity to conduct defibrillation, the catheter comprising
an insulated tube member having a multi-lumen structure, a handle connected to a proximal end of the tube member, a first electrode group composed of a plurality of ring-like electrodes and installed in a distal region of the tube member,
a second electrode group composed of a plurality of ring-like electrodes and installed on the tube member towards proximal direction from the first electrode group,
a first lead wire group composed of lead wires connected to the respective electrodes making up the first electrode group, and
a second lead wire group composed of lead wires connected to the respective electrodes making up the second electrode group, wherein the first lead wire group and the second lead wire group respectively extend into different lumens of the tube member, and
voltages different in polarity from each other are respectively applied to the first electrode group and the second electrode group when defibrillation is conducted.

2. The intracardiac defibrillation catheter according to claim 1, which comprises
a potential-measuring electrode group composed of a plurality of electrodes and installed on the tube member apart from the first electrode group or the second electrode group, and
a lead wire group for potential measurement, which is composed of lead wires connected to the respective electrodes making up the potential-measuring electrode group, wherein
the lead wire group for potential measurement extends into any other lumen of the tube member than both lumens into which the first lead wire group and the second lead wire group extend.

3. The intracardiac defibrillation catheter according to claim 1, which comprises
a distal-side potential-measuring electrode group composed of a plurality of electrodes and installed on the tube member towards distal direction from the first electrode group,
a proximal-side potential-measuring electrode group composed of a plurality of ring-like electrodes and installed on the tube member towards proximal direction from the second electrode group,
a third lead wire group composed of lead wires connected to the respective electrodes making up the distal-side potential-measuring electrode group, and
a fourth lead wire group, composed of lead wires connected to the respective electrodes making up the proximal-side potential-measuring electrode group,
wherein the third lead wire group and the fourth lead wire group extend into any other lumen of the tube member than both lumens into which the first lead wire group and the second lead wire group extend.

4. The intracardiac defibrillation catheter according to claim 3, wherein 4 lumens are formed in the tube member,
the first lead wire group extends into a first lumen,
the second lead wire group extends into a second lumen, the third lead wire group and fourth lead wire group extend into a third lumen, and
a pull wire for distal end deflection operation extends into a fourth lumen.

5. The intracardiac defibrillation catheter according to any one of claims 1 to 4, wherein the first lead wire group and the second lead wire group extend into inner holes of insulated tubes different from each other in the interior of the handle.

6. The intracardiac defibrillation catheter according to claim 4, wherein in the interior of the handle,
the first lead wire group extends into a first insulated tube connected to the first lumen,
the second lead wire group extends into a second insulated tube connected to the second lumen, and
the third lead wire group and the fourth lead wire group extend into a third insulated tube connected to the third lumen.

7. The intracardiac defibrillation catheter according to any one of claims 1 to 4, which is inserted into a cardiac cavity to remove atrial fibrillation caused during cardiac catheterization.

8. The intracardiac defibrillation catheter according to claim 5, which is inserted into a cardiac cavity to remove atrial defibrillation caused during cardiac catheterization.

9. The intracardiac defibrillation catheter according to claim 6, which is inserted into a cardiac cavity to remove atrial defibrillation cause during cardiac catheterization.
